(19) United States
(12) Patent Application Publication
(10) Pub. No.: US 2006/0025476 A1
(43) Pub. Date: Feb. 2, 2006

(54) Use of methyl pyruvate for the purpose of reducing weight gain in mammals.

(75) Inventors: Stanley Charles Antosh, Palm Springs, CA (US); Anthony J. Meduri, New York City, NY (US)

Correspondence Address: ROZSA LAW GROUP LC, 15910 VENTURA BOULEVARD, SUITE 1601, ENCINO, CA, 91436-2815, US

(73) Assignees: Stanley Charles Antosh, Palm Springs, CA (US); Anthony J Meduri, New York City, NY (US)

(21) Appl. No.: 10/710,830

(22) Filed: Aug. 5, 2004

Related U.S. Application Data
(63) Continuation-in-part of application No. 10/710,710, filed on Jul. 29, 2004.

(51) Int. Cl. A61K 31/22

(52) U.S. Cl. 514/546

(57) ABSTRACT

The present invention relates to the use of methyl pyruvic acid (a methyl ester of pyruvic acid) and/or methyl pyruvate (methyl pyruvate is the ionized form of methyl pyruvic acid) for the purpose of reducing weight (fat) gain in mammals by orally administering therapeutically effective amounts of methyl pyruvate. The method also has the effect of increasing body protein concentration, improving insulin resistance, lower fasting insulin levels, preventing fat deposition and increasing cellular energy production. When used as a dietary supplement, energizer or pharmaceutical, this anion can be formulated as a salt. The methyl pyruvate compounds which can be used in the present method include: (1) a salt using a monovalent cation (such as sodium or potassium methyl pyruvate) or (2) a divalent cation (such as calcium or magnesium methyl pyruvate) and analogs of these compounds which can act as substrates or substrate analogs for methyl pyruvate Use of methyl pyruvate and/or methyl pyruvic acid can be effective when administered orally or infused on either a chronic and/or acute basis. In the following text, the terms “methyl pyruvate, methyl pyruvate compounds, methyl pyruvic acid”are used interchangeably.

### BACKGROUND OF INVENTION

Current U.S. Class: 514/251; 514/557; 514/909 International Class: A61K 031/19 Field of Search: 424/252,317

References Cited [Referenced By] U.S. Pat. No. 3,838,027 September, 1974 Berntsson 204/74. U.S. Pat. No. 4,197,316 April, 1980 Yu et al. 424/317. U.S. Pat. No. 4,296,127 October, 1981 Walser 424/319. U.S. Pat. No. 4,351,835 September, 1982 Stanko 514/251. U.S. Pat. No. 4,548,937 October, 1985 Stanko 514/251. U.S. Pat. No. 4,734,276 March, 1988 Ziegler 233/265. U.S. Pat. No. 4,874,790 October, 1989 Stanko 514/557. U.S. Pat. No. 5,053,527 October, 1991 Christidis et al. 560/174. U.S. Pat. No. 5,089,492 February, 1992 Gerling et al. 514/210.

### OTHER REFERENCES

Prevention of Alcohol-induced Fatty Liver By Natural Metabolites and Riboflavin”, Stanko et al., Journal of Laboratory and Clinical Medicine, February 1978.

A Facile Preparation of Primary Carboxamides”, Pellegata et al., Communications, May 1985, pp. 517-519.

Inhibition of Lipid Accumulation and Enhancement of Energy Expenditure by the Addition of Pyruvate and Dihydroxyacetone to a Rat Diet”, Stanko et al. Metabolism, vol. 35, No. 2, February, 1986, pp. 182-186.

Howlett R A, Gonzalez N C, Wagner H E, Fu Z, Britton S L, Koch L G, Wagner P D. Genetic Models in Applied Physiology: Selected Contribution: Skeletal muscle capillarity and enzyme activity in rats selectively bred for running endurance. J Appl Physiol. 2003 April; 94 (4):1682-8.

Henderson K K, Wagner H, Favret F, Britton S L, Koch L G, Wagner P D, Gonzalez N C. Determinants of maximal O(2) uptake in rats selectively bred for endurance running capacity. J Appl Physiol. 2002 October; 93 (4): 1265-74.

Hussain S O, Barbato J C, Koch L G, Mettig P J, Britton S L. function in rats selectively bred for low- and high-capacity running. Am J Physiol Regul Integr Comp Physiol. 2001 December; 281 (6):R1787-91.

Greiwe J S, Hickner R C, Hansen P A, Racette S B, Chen M M, Holloszy J O. Effects of endurance exercise training on muscle glycogen accumulation in humans. J Appl Physiol. 1999 July; 87 (1):222-6.

Kayser B, Hoppeler H, Desplanches D, Marconi C, Broers B, Cerretelli P. Muscle ultrastructure and biochemistry of lowland Tibetans. J Appl Physiol. 1996 July; 81 (1):419-25.

Bussieres L M, Pflugfelder P W, Taylor A W, Noble E G, Kostuk W J. in skeletal muscle morphology and biochemistry after cardiac transplantation. Am J Cardiol. 1997 Mar. 1; 79 (5):630-4.

Roberts K C, Nixon C, Unthank J L, Lash J M. artery ligation stimulates capillary growth and limits training—induced increases in oxidative capacity in rats. Microcirculation. 1997 June; 4 (2):253-60.

Sexton W L. Vascular adaptations in rat hindlimb skeletal muscle after voluntary running-wheel exercise. J Appl Physiol. 1995 July; 79 (1):287-96.

McAllister R M, Reiter B L, Amann J F, Laughlin M H. Skeletal muscle biochemical adaptations to exercise training in miniature swine. J Appl Physiol. 1997 June; 82 (6):1862-8.

SL, Rennie C D, Hamilton S J, Tarnopolsky. Changes in skeletal muscle in males and females following endurance training. Can J Physiol Pharmacol. 2001 May; 79 (5):386-92.

AR, Spina R J, King D S, Rogers M A, Brown M, Nemeth P M, Holloszy J O. Skeletal muscle adaptations to endurance training in 60-70-yr-old men and women. J Appl Physiol. 1992 ay; 72 (5):1780-6.

N, Torres S H, Rivas M. Inactivity changed fiber type proportion and capillary supply in cat muscle. Comp Biochem Physiol A Physiol. 1997 June; 117 (2):211-7.

Y, Shimegi S, Masuda K, Sakato H, Ohmori H, Katsuta S. Effects of different intensity endurance training on the capillary network in rat skeletal muscle. Int J Microcirc Clin Exp. 1997 March-April; 17 (2):93-6.

D, Fraga C, Laughlin M H, Amann J F. Regional changes in capillary supply in skeletal muscle of high-intensity endurance-trained rats. J Appl Physiol. 1996 August; 81 (2):619-26.

RH, Booth F W, Winder W W, Holloszy J O. Skeletal muscle respiratory capacity, endurance, and glycogen utilization. Am J. Physiol. 1975 April; 228 (4):1029-33.

MG, Costill D L, Kirwan J P, Fink W J, Dengel D R. Muscle fiber composition and respiratory capacity in triathletes. Int J Sports Med. 1987 December; 8 (6):383-6.

JR, Coyle E F, Osbakken M. of heart failure on skeletal muscle in dogs. Am J. Physiol. 1992 April; 262 (4 Pt 2):H993-8.

M, Eriksson B O, Lonn L, Rundqvist B, Sunnerhagen K S, Swedberg K. Skeletal muscle characteristics, muscle strength and thigh muscle area in patients before and after cardiac transplantation. Eur J Heart Fail. 2001 January; 3 (1): 59-67.

RT, Hogan M C, Stary C, Bebout D E, Mathieu-Costello O, Wagner P D. Structural basis of muscle O(2) diffusing capacity: evidence from muscle function in situ. J Appl Physiol. 2000 February; 88 (2):560-6.

Goreham C, Green H J, Ball-Burnett M, Ranney D. High-resistance training and muscle metabolism during prolonged exercise. Am J. Physiol. 1999 March; 276 (3 Pt 1): E489-96.

WL, Laughlin M H. Influence of endurance exercise training on distribution of vascular adaptations in rat skeletal muscle. Am J. Physiol. 1994 February; 266 (2 Pt 2):H483-90.

DR, Gregorevic P, Warmington S A, Williams D A, Lynch G S. Endurance training adaptations modulate the redox-force relationship of rat isolated slow-twitch skeletal muscles. Clin Exp Pharmacol Physiol. 2003 January-February; 30 (1-2):77-81.

AX, Brunet A, Guezennec C Y, Monod H. Skeletal muscle changes after endurance training at high altitude. J Appl Physiol. 1991 December; 71 (6):2114-21.

RJ, Chi M M, Hopkins M G, Nemeth P M, Lowry O H, Holloszy J O. Mitochondrial enzymes increase in muscle in response to 7-10 days of cycle exercise. J Appl Physiol. 1996 June; 80 (6):2250-4.

Coggan A R, Spina R J, Rogers M A, King D S, Brown M, Nemeth P M, Holloszy J O. Histochemical enzymatic characteristics of skeletal muscle in master athletes. J Appl Physiol. 1990 May; 68 (5):1896-901.

JS, Bruce C R, Spriet L L, Hawley J A. Interaction of diet and training on endurance performance in rats. Exp Physiol. 2001 July; 86 (4):499-508.

Sumida K D, Donovan C M. Endurance training fails to inhibit skeletal muscle glucose uptake during exercise. J Appl Physiol. 1994 May; 76 (5): 1876-81.

Sullivan M J, Green H J, Cobb F R. Skeletal muscle biochemistry and histology in ambulatory patients with long-term heart failure. Circulation. 1990 February; 81 (2):518-27.

DM, Coyle E, Coggan A, Beltz J, Ferraro N, Montain S, Wilson J R. Contribution of intrinsic skeletal muscle changes to 31P NMR skeletal muscle metabolic abnormalities in patients with chronic heart failure. Circulation. 1989 November; 80 (5): 1338-46.

M, Nakano H, Higaki Y, Nakamura T, Katsuta S, Kumagai S. Increased wheel-running activity in the genetically skeletal muscle fast-twitch fiber-dominant rats. J Appl Physiol. 2003 January; 94 (1):185-92.

DL, Fink W J, Getchell L H, Ivy J L, Witzmann F A. Lipid metabolism in skeletal muscle of endurance-trained males and females. J Appl Physiol. 1979 October; 47 (4):787-91.

Coggan A R, Spina R J, Kohrt W M, Holloszy J O. Effect of prolonged exercise on muscle citrate concentration before and after endurance training in men. Am J. Physiol. 1993 February; 264 (2 Pt 1):E215-20.

Snyder G K. Capillary growth in chick skeletal muscle with normal maturation and hypertrophy. Respir Physiol. 1995 December; 102 (2-3):293-301.

Weston A R, Wilson G R, Noakes T D, Myburgh K H. Skeletal muscle buffering capacity is higher in the superficial vastus than in the soleus of spontaneously running rats. Acta Physiol Scand. 1996 June; 157 (2):211-6.

Torgan C E, Brozinick J T Jr, Kastello G M, Ivy J L Muscle morphological and biochemical adaptations to training in obese Zucker rats. J Appl Physiol. 1989 November; 67 (5): 1807-13.

Sexton W L, Poole D C, Mathieu-Costello 0. Microcirculatory structure-function relationships in skeletal muscle of diabetic rats. Am J. Physiol. 1994 April; 266 (4 Pt 2):H1502-11.

Parsons D, Musch T I, Moore R L, Haidet G C, Ordway G A. Dynamic exercise training in foxhounds. II. Analysis of skeletal muscle. J Appl Physiol. 1985 July; 59 (1):190-7.

Celsing F, Blomstrand E, Melichna J, Terrados N, Clausen N, Lins P E, Jansson E. Effect of hyperthyroidism on fibre-type composition, fibre area, glycogen content and enzyme activity in human skeletal muscle. Clin Physiol. 1986 April; 6 (2): 171-81.

Coggan A R, Abduljalil A M, Swanson S C, Earle M S, Farris J W, Mendenhall L A, Robitaille P M. Muscle metabolism during exercise in young and older untrained and endurance-trained men. J Appl Physiol. 1993 November; 75 (5):2125-33.

Leon-Velarde F, Sanchez J, Bigard A X, Brunet A, Lesty C, Monge C. High altitude tissue adaptation in Andean coots: capillarity, fibre area, fibre type and enzymatic activities of skeletal muscle. J Comp Physiol [B]. 1993; 163 (1):52-8.

Maxwell L C, White T P, Faulkner J A. Oxidative capacity, blood flow, and capillarity of skeletal muscles. J Appl Physiol. 1980 October; 49 (4):627-33.

Foster C, Costill D L, Daniels J T, Fink W J. Skeletal muscle enzyme activity, fiber composition and V02 max in relation to distance running performance. Eur J Appl Physiol Occup Physiol. 1978 Aug. 15; 39 (2):73-80.

Mitchell M L, Byrnes W C, Mazzeo R S. A comparison of skeletal muscle morphology with training between young and old Fischer 344 rats. Mech Ageing Dev. 1991 Apr. 1; 58 (1):21-35.

Bigard A X, Brunet A, Guezennec C Y, Monod H. Effects of chronic hypoxia and endurance training on muscle capillarity in rats. Pflugers Arch. 1991 October; 419 (3-4):225-9.

Thomas D P, Jenkins R R. Effects of beta 1-vs. beta 1-beta 2-blockade on training adaptations in rat skeletal muscle. J Appl Physiol. 1986 May; 60 (5):1722-6.

Duscha B D, Annex B H, Keteyian S J, Green H J, Sullivan M J, Samsa G P, Brawner C A, Schachat F H, Kraus W E. Differences in skeletal muscle between men and women with chronic heart failure. J Appl Physiol. 2001 January; 90 (1):280-6.

Bangsbo J, Michalsik L, Petersen A. Accumulated O₂ deficit during intense exercise and muscle characteristics of elite athletes. Int J Sports Med. 1993 May; 14 (4):207-13.

Tanaka T, Ohira Y, Danda M, Hatta H, Nishi I. Improved fatigue resistance not associated with maximum oxygen consumption in creatine-depleted rats. J Appl Physiol. 1997 June; 82 (6): 1911-7.

Hammeren J, Powers S, Lawler J, Criswell D, Martin D, Lowenthal D, Pollock M. skeletal muscle oxidative and antioxidant enzyme activity in senescent rats. Int J Sports Med. 1992 Jul; 13 (5):412-6.

MacDougall J D, Hicks A L, MacDonald J R, McKelvie R S, Green H J, Smith K M. Muscle performance and enzymatic adaptations to sprint interval training. J Appl Physiol. 1998 June; 84 (6):2138-42.

Schluter J M, Fitts R H. Shortening velocity and ATPase activity of rat skeletal muscle fibers: effects of endurance exercise training. Am J. Physiol. 1994 June; 266 (6 Pt 1):C1699-73.

Suter E, Hoppeler H, Claassen H, Billeter R, Aebi U, Horber F, Jaeger P, Marti B. Ultrastructural modification of human skeletal muscle tissue with 6-month moderate-intensity exercise training. Int J Sports Med. 1995 April; 16 (3):160-6.

Russell J A, Kindig C A, Behnke B J, Poole D C, Musch T I. Effects of aging on capillary geometry and hemodynamics in rat spinotrapezius muscle. Am J Physiol Heart Circ Physiol. 2003 Mar. 20.

Magnusson G, Kaijser L, Rong H, Isberg B, Sylven C, Saltin B. capacity in heart failure patients: relative importance of heart and skeletal muscle. Clin Physiol. 1996 March; 16 (2): 183-95.

Frandsen U, Hoffner L, Betak A, Saltin B, Bangsbo J, Hellsten Y. training does not alter the level of neuronal nitric oxide synthase in human skeletal muscle. J Appl Physiol. 2000 September; 89 (3):1033-8.

Chati Z, Michel C, Escanye J M, Mertes P M, Ribuot C, Canet D, Zannad F. Skeletal muscle beta-adrenoreceptors and phosphate metabolism abnormalities in heart failure in rats. Am J Physiol. 1996 November; 271 (5 Pt 2):H1739-45.

Snyder G K. Capillarity and diffusion distances in skeletal muscles in birds. J Comp Physiol [B]. 1990; 160 (5):583-91.

Lambert M I, Van Zyl C, Jaunky R, Lambert E V, Noakes T D. Tests of running performance do not predict subsequent spontaneous running in rats. Physiol Behav. 1996 July; 60 (1): 171-6.

Tikkanen H O, Naveri H K, Harkonen M H. Alteration of regulatory enzyme activities in fast-twitch and slow-twitch muscles and muscle fibres in low-intensity endurance-trained rats. Eur J Appl Physiol Occup Physiol. 1995; 70 (4):281-7.

Moore R L, Gollnick P D. Response of ventilatory muscles of the rat to endurance training. Pflugers Arch. 1982 January; 392 (3):268-71.

Hickson R C, Heusner W W, Van Huss W D. Skeletal muscle enzyme alterations after sprint and endurance training. J Appl Physiol. 1976 June; 40 (6):868-71.

Hickner R C, Fisher J S, Hansen P A, Racette S B, Mier C M, Turner M J, Holloszy J O. Muscle glycogen accumulation after endurance exercise in trained and untrained individuals. J Appl Physiol. 1997 September; 83 (3):897-903.

Zhan W Z, Swallow J G, Garland T Jr, Proctor D N, Carter P A, Sieck G C. Effects of genetic selection and voluntary activity on the medial gastrocnemius muscle in house mice. J Appl Physiol. 1999 December; 87 (6):2326-33. Snyder G K, Wilcox E E, Burnham E W. Effects of hypoxia on muscle capillarity in rats. Respir Physiol. 1985 October; 62 (1):135-40.

Coyle E F, Coggan A R, Hopper M K, Walters T J. Determinants of endurance in well-trained cyclists. J Appl Physiol. 1988 June; 64 (6):2622-30.

Baldwin K M, Cooke D A, Cheadle W G. Time course adaptations in cardiac and skeletal muscle to different running programs. J Appl Physiol. 1977 February; 42 (2):267-72.

Howlett R A, Heigenhauser G J, Hultman E, Hollidge-Horvat M G, Spriet L L. Effects of dichloroacetate infusion on human skeletal muscle metabolism at the onset of exercise. Am J Physiol. 1999 July; 277 (1 Pt 1):E18-25.

Jansson E, Sylven C. of key enzymes in the energy metabolism of human myocardial and skeletal muscle. Clin Physiol. 1986 October; 6 (5):465-71.

Baldwin K M, Winder W W, Holloszy J O. Adaptation of actomyosin ATPase in different types of muscle to endurance exercise. Am J. Physiol. 1975 August; 229 (2):422-6.

Bigard A X, Brunet A, Serrurier B, Guezennec C Y, Monod H. of endurance training at high altitude on diaphragm muscle properties. Pflugers Arch. 1992 December; 422 (3):239-44.

Kalliokoski K K, Kuusela T A, Laaksonen M S, Knuuti J, Nuutila P. Muscle fractal vascular branching pattern and microvascular perfusion heterogeneity in endurance-trained and untrained men. J Physiol. 2003 Jan. 15; 546 (Pt 2):529-35.

Saltin B, Kim C K, Terrados N, Larsen H, Svedenhag J, Rolf C J. Morphology, enzyme activities and buffer capacity in leg muscles of Kenyan and Scandinavian runners. Scand J Med Sci Sports. 1995 August; 5 (4):222-30.

Maltais F, LeBlanc P, Simard C, Jobin J, Berube C, Bruneau J, Carrier L, Belleau R. Skeletal muscle adaptation to endurance training in patients with chronic obstructive pulmonary disease. Am J Respir Crit Care Med. 1996 August; 154 (2 Pt 1):442-7.

Green H J, Jones S, Ball-Burnett M E, Smith D, Livesey J, Farrance B W. Early muscular and metabolic adaptations to prolonged exercise training in humans. J Appl Physiol. May; 70 (5):2032-8.

Snyder G K, Farrelly C, Coelho J R. Adaptations in skeletal muscle capillarity following changes in oxygen supply and changes in oxygen demands. Eur J Appl Physiol Occup Physiol. 1992; 65 (2):158-63.

Green H, Roy B, Grant S, Otto C, Pipe A, McKenzie D, Johnson M. Human skeletal muscle exercise metabolism following an expedition to mount denali. Am J Physiol Regul Integr Comp Physiol. 2000 November; 279 (5):R1872-9.

Gosselin L E, Betlach M, Vailas A C, Thomas D P. Training-induced alterations in young and senescent rat diaphragm muscle. J Appl Physiol. 1992 April; 72 (4):1506-11.

Wang X N, Williams T J, McKenna M J, Li J L, Fraser S F, Side E A, Snell G I, Walters E H, Carey M F. Skeletal muscle oxidative capacity, fiber type, and metabolites after lung transplantation. Am J Respir Crit Care Med. 1999 July; 160 (1):57-63.

Riedy M, Moore R L, Gollnick P D. Adaptive response of hypertrophied skeletal muscle to endurance training J Appl Physiol. 1985 July; 59 (1):127-31.

Miller W C, Bryce G R, Conlee R K. Adaptations to a high-fat diet that increase exercise endurance in male rats. J Appl Physiol. 1984 January; 56 (1):78-83.

W M, Costill D L, Fink W J, Hagerman F C, Armstrong L E, Murray T F. Effect of a 42.2-km footrace and subsequent rest or exercise on muscle glycogen and enzymes. J Appl Physiol. 1983 October; 55 (4):1219-24.

Baldwin K M, Hooker A M, Herrick R E, Schrader L F. Respiratory capacity and glycogen depletion in thyroid-deficient muscle. J Appl Physiol. 1980 July; 49 (1): 102-6.

Willis W T, Brooks G A, Henderson S A, Dallman P R. Effects of iron deficiency and training on mitochondrial enzymes in skeletal muscle. J Appl Physiol. 1987 June; 62 (6):2442-6.

McConell G, McCoy M, Proietto J, Hargreaves M. Skeletal muscle GLUT-4 and glucose uptake during exercise in humans. J Appl Physiol. 1994 September; 77 (3): 1565-8.

Nakatani A, Han D H, Hansen P A, Nolte L A, Host H H, Hickner R C, Holloszy J O. Effect of endurance exercise training on muscle glycogen supercompensation in rats. J Appl Physiol. 1997 February; 82 (2):711-5.

RM, Terjung R L. Training-induced muscle adaptations: increased performance and oxygen consumption. J Appl Physiol. 1991 April; 70 (4):1569-74.

AT, Foley J M, Meyer R A. Linear dependence of muscle phosphocreatine kinetics on oxidative capacity. Am J. Physiol. 1997 February; 272 (2 Pt 1):C501-10.

S, Powers S K, Lawler J, Criswell D, Dodd S, Edwards W. Endurance training-induced increases in expiratory muscle oxidative capacity. Med Sci Sports Exerc. 1992 May; 24 (5):551-5.

PA, Waldmann M L, Meyer W L, Brown K A, Poehlman E T, Pendlebury W W, Leslie K O, Gray P R, Lew R R, LeWinter M M. Skeletal muscle and cardiovascular adaptations to exercise conditioning in older coronary patients. Circulation. 1996 Aug. 1; 94 (3):323-30.

VP, Gettelman G J, Widrick J J, Fitts R H. Substrate and enzyme profile of fast and slow skeletal muscle fibers in rhesus monkeys. J Appl Physiol. 1999 January; 86 (1):335-40.
p, Garland T Jr, Swallow J G, Guderley H. Effects of voluntary activity and genetic selection on muscle metabolic capacities in house mice Mus domesticus. J Appl Physiol. 2000 October; 89 (4):1608-16.

JL, Serrano A L, Henckel P. Activities of selected aerobic and anaerobic enzymes in the gluteus mediusmuscle of endurance horses with different performance recordsVet Rec. 1995 Aug. 19; 137 (8):187-92.

Apple F S, Rogers M A. Skeletal muscle lactate dehydrogenase isozyme alterations in men and women marathon runners. J Appl Physiol. 1986 August; 61 (2):477-81.

Diaz-Herrera P, Torres A, Morcuende J A, Garcia-Castellano J M, Calbet J A, Sarrat R. Effect of endurance running on cardiac and skeletal muscle in rats. Histol Histopathol. 2001 January; 16 (1):29-35.

Soar P K, Davies C T, Fentem P H, Newsholme E A. effect of endurance-training on the maximum activities of hexokinase, 6-phosphofructokinase, citrate synthase, and oxoglutarate dehydrogenase in red and white muscles of the rat. Biosci Rep. 1983 September; 3 (9):831-5.

Goodpaster B H, He J, Watkins S, Kelley D E Skeletal muscle lipid content and insulin resistance: evidence for a paradox in endurance-trained athletes. J Clin Endocrinol Metab. 2001 December; 86 (12):5755-61.

Ripoll E, Sillau A H, Banchero N. Changes in the capillarity of skeletal muscle in the growing rat. Pflugers Arch. 1979 Jun. 12; 380 (2):153-8.

Kirwan J P, Costill D L, Flynn M G, Neufer P D, Fink W J, Morse W M. Effects of increased training volume on the oxidative capacity, glycogen content and tension development of rat skeletal muscle. Int J Sports Med. 1990 December; 11 (6):479-83.

Wallberg-Henriksson H, Gunnarsson R, Henriksson J, Ostman J, Wahren J. Influence of physical training on formation of muscle capillaries in type I diabetes. Diabetes. 1984 September; 33 (9):851-7.

### FIELD OF THE INVENTION

The present invention relates to the field of obesity and the use of methyl pyruvic acid (a methyl ester of pyruvic acid) and/or methyl pyruvate (methyl pyruvate is the ionized form of methyl pyruvic acid) for the purpose of reducing weight (fat) gain in mammals by infusing or orally administering therapeutically effective amounts of methyl pyruvate. The method also has the effect of increasing body protein concentration, improving insulin resistance, lower fasting insulin levels, preventing fat deposition and increasing cellular energy production. When used as a dietary supplement, energizer or pharmaceutical, this anion can be formulated as a salt.

Use of methyl pyruvate and/or methyl pyruvic acid can be effective when administered orally or infused on either a chronic and/or acute basis. In the following text, the terms “methyl pyruvate, methyl pyruvate compounds, methyl pyruvic acid” are used interchangeably.

Obesity is a complex disorder characterized by the accumulation of excess adipose tissue. While obesity has long been considered a behavioral disorder, discovery of the hormone leptin in 1994 catalyzed the field of obesity research by demonstrating the existence of anafferent humoral signal from adipose tissue to the central nervous system. Current evidence suggests that once adipose tissue accumulates, a system of overlapping neuroendocrine systems prevents it from diminishing. This counter-regulatory mechanism, which has probably evolved as protection against starvation and fetal or neonatal wastage, causes changes in appetite and metabolism that make volitional weight loss difficult to achieve.

Obesity is defined in terms of BMI, calculated as weight (kg)/[height (m)]2. Although a continuous variable, BMI has been categorized based on epidemiologic data to denote the relative risk of developing comorbid conditions. A BMI less than 25 is considered to be normal, 25-29.9 is overweight, and greater than or equal to 30, obese. Data from the 1999 National Health Nutritional and Exercise survey demonstrated that 34% of adults in the United States were over-weight, and 30.8% obese, resulting in a total of 64.8% above normal weight. The prevalence of overweight and obesity in children was 13%, a doubling since 1980, while adolescents have experienced a tripling in prevalence since then.

Being overweight or obese substantially increases the risk of morbidity from a number of conditions, including type 2 diabetes mellitus (DM), hypertension, dyslipidemia, coronary heart disease, congestive heart failure, stroke, gallbladder disease, hepatic steatosis, osteoarthritis, sleep apnea, and endometrial, breast, prostate, and colon cancers. An increase in all-cause mortality is also associated with higher body weights. Adipose tissue is an active endocrine organ that produces free fatty acids (metabolized through the beta-oxidation cycle), hormones, such as IL-6, TNF-α, plasminogen activation inhibitor1, angiotensinogen, and others, directly related to the insulin resistance, hyperlipidemia, inflammation, thrombosis, and hypertension that characterize obesity.

Intracellular metabolites also regulate energy metabolism and may signal the availability of fuel to metabolite-sensitive hypothalamic neurons. Interference with central pathways involved in the synthesis of malonyl-CoA or fatty acids with either genetic knockouts of acetyl-CoA carboxylase or fatty acid synthase inhibitors have been shown to decrease body fat. It has also been shown that an adipocyte-derived hormone, Acrp30 (AdipoQ or adiponectin), increases fatty acid oxidation (beta-oxidation) in muscle and liver and may regulate fat accumulation without significantly affecting food intake. Many other molecules, including other peptides, neurotransmitters, cytokines, steroid hormones, enzymes, and peroxisome proliferator-activated receptor (PPAR) agonists affect energy homeostasis.

The high frequency of relapse and limited weight loss attainable with non-surgical therapies are among the frustrations experienced by the obese patient. Investigation of the energetics of weight loss and weight gain demonstrates that maintenance of a reduced body weight is associated with declines in energy expenditure that are greater than can be accounted for by reductions in metabolic mass. The weight-reduced state is also characterized by reduced sympathetic and increased parasympathetic nervous tone. These changes persist for months to years in weight-reduced subjects.

Non-pharmacological treatments for obesity include behavior therapy, exercise, and calorie-restricted diets. The goal of behavior therapy is to overcome barriers to compliance with a diet and physical activity regimen. Physical activity increases energy expenditure and is a key component of any weight maintenance program, counteracting the reduction in total energy expenditure that occurs with weight loss. In order to induce weight loss a calorie deficit must be created.

U.S. Pat. No. 4,351,835 teaches a method for preventing body fat deposition in mammals by oral administration of a mixture of pyruvate and dihydroxyacetone (DHA). Subsequent additional research with rats investigated the effect of pyruvate and DHA under normal dietary conditions. In that study, rats were fed either a controlled diet or an experimental diet in which part of the carbohydrates were replaced with a 1:1 mixture of pyruvate and DHA, which mixture constituted 15% of the total caloric intake. Rats which received the experimental diet gained less weight, and had greater rates of heat reduction and energy expenditure than rats receiving a control diet. The experimental diet reduced body fat content by 32% without any significant effect on either protein or water content. Similarly, in another study, Type II diabetic humans were fed 56 grams of pyruvate and DHA in a 1:1 mixture for seven days, during which time period glucose tolerance and turnover were measured. Reductions in fasting blood glucose concentration and peak glucose concentration after a glucose tolerance test were observed.

Yet another study assessed the relative effectiveness of pyruvate and DHA. In that study, obese Zucker rats were placed in one of four diet groups. One diet was a control and each of the other diets featured a semi-purified rat diet with only one of the following features: (a) 6% pyruvate, (b) 6% DHA, or (c) 6% pyruvate/DHA (1:1). A number of physiologic variables were measured.

The conclusion of the study was that generally changes due to the addition of DHA or pyruvate/DHA to the diet were not as great as changes due to the addition of only pyruvate. In fact, often the changes due to the addition of DHA or pyruvate/DHA could either be attributed to feed restriction or to the pyruvate in combination.

U.S. Pat. No. 4,548,937 discloses a method for minimizing weight gain by adding pyruvate to the diet. Based on the above studies, the experimental data indicated that pyruvate was an efficacious compound in altering metabolic variables in rats.

A problem exists in administering effective dosages of pyruvate to humans in that heretofore the only ways to supply pyruvate have been in the form of a liquified pyruvic acid or in the form of the mineral salts of pyruvate, for example via sodium, potassium or calcium salts. These salts are organoleptically poor, as is tolerance of these salts. Furthermore, in humans the amount of these salts required to obtain the proper dosage of pyruvate for maximal effect raises the electrolyte level of the recipient to 2-6 times the safe and adequate recommended level when given as a supplement to a typical diet. With respect to the liquid pyruvic acid, the liquid is very acidic and results in the body literally being burned. Attempting to solve the acidity problem through dilution results in the human body being unable to ingest acceptable levels of pyruvate. It is thus apparent that the need exists for an improved method of addressing obesity and the associated health risks by administering methyl pyruvate to humans.

Although pyruvate theoretically appears to be an efficacious compound in addressing obesity and problems associated with Type II diabetes, the utility of pyruvate in humans in the clinical management of Type II diabetes or obesity has been limited by the elevated mineral load associated with pyruvate salts, which until this time were the only practical method of supplying pyruvate to the body. Pyruvate can also be supplied as a liquid acid, but it is so acidic that it must be diluted. When the liquid acid is diluted sufficiently to be tolerable, it requires too large a volume of liquid to be consumed in order to obtain a sufficient ingestion of pyruvate. Table I illustrates the raised electrolyte levels associated with the generation of an effective dose (28 grams) of pyruvate in the form of pyruvate salts, with the salts being used either alone or in combination.

TABLE 1

| 28 g Pyruvate | Na+ | K+ | Ca++ |
|---|---|---|---|
| Single salt, mg | 7,000 | — | — |
| Single salt, mg | — | 12,560 | — |
| Single salt, mg | — | — | 6,400 |
| Combination*, mg | 3,500 | — | 3,200 |
| Combination*, mg | — | 6,300 | 3,200 |
| Combination*, mg | 2,330 | 4,180 | 2,140 |
| ESADDI** range, mg | 1,100-3,300 | 1,875-5,625 | 1,200 |

*Each salt is added as an equal proportion of the total 28 grams of pyruvate.
**Estimated Safe and Adequate Daily Dietary Intake in the RDA 10th edition.

As can be seen from Table I, the electrolyte level is raised to between 2-6 times the level recommended in humans, regardless of how the pyruvate salts are ingested.

### SUMMARY OF INVENTION

The present invention relates the use of methyl pyruvate as a dietary supplement, which can be utilized by obese or overweight mammals for the reduction of weight. The present invention further relates to the field of cellular energy production and more particularly to enhancing the production of the energy by utilizing methyl pyruvate compounds, which modulate the system. This modulation also has the effect of increasing body protein concentration, improving insulin resistance, lower fasting insulin levels, preventing fat deposition and increasing cellular energy production. When used as a dietary supplement, energizer or pharmaceutical, this anion can be formulated as a salt.

A preferred mode of use involves co-administration of a methyl pyruvate salt along with one or more agents that promote energy. Typical dosages of methyl pyruvate compounds will depend on factors such as size, weight, age, health and fitness level.

The present invention further pertains to methods of use of methyl pyruvate compounds in combination with vitamins, coenzymes, mineral substances, amino acids, herbs, antioxidants, metabolic compounds and creatine compounds, which act on the cells for enhancing energy production and expenditure, thus the ability and desire to be active.

### DETAILED DESCRIPTION

The present invention relates the use of methyl pyruvate as a dietary supplement, which can be utilized by obese or overweight mammals for the reduction of weight. Methyl pruvate is the ionized form of methyl pyruvic acid (CH3C(O)CO2CH3). At physiologic pH, the hydrogen proton dissociates from the carboxylic acid group, thereby generating the methyl pyruvate anion. When used as a pharmaceutical or dietary supplement, this anion can be formulated as a salt, using a monovalent or divalent cation such as sodium, potassium, magnesium, or calcium.

Cells require energy to survive and perform their physiological functions, and it is generally recognized that the only source of energy for cells is the glucose and oxygen delivered by the blood. There are two major components to the process by which cells utilize glucose and oxygen to produce energy. The first component entails anaerobic conversion of glucose to pyruvate, which releases a small amount of energy, and the second entails oxidative conversion of pyruvate to carbon dioxide and water with the release of a large amount of energy. Pyruvate is continuously manufactured in the living organism from glucose. The process by which glucose is converted to pyruvate involves a series of enzymatic reactions that occur anaerobically (in the absence of oxygen). This process is called “glycolysis”. A small amount of energy is generated in the glycolytic conversion of glucose to pyruvate, but a much larger amount of energy is generated in a subsequent more complicated series of reactions in which pyruvate is broken down to carbon dioxide and water. This process, which does require oxygen and is referred to as “oxidative respiration”, involves the stepwise metabolic breakdown of pyruvate by various enzymes of the Krebs tricarboxylic acid cycle and conversion of the products into high-energy molecules by electron transport chain reactions.

The enzyme, pyruvate dehydrogenase (PDH), catalyzes the conversion of pyruvate to acetyl CoA, a pivotal reaction in glucose metabolism. In the mitochondrial matrix, decreased free CoA, relative to acetyl CoA, inhibits the activity of PDH. Carnitine acetyl-transferase (CAT) catalyzes the transfer of the acetyl group from acetyl CoA to L-carnitine, freeing CoA to participate in the PDH reaction. Acetyl-L-carnitine can be exported from the mitochondria through the activity of CAT. Within the mitochondrial matrix, short- and medium-chain fatty acids can be transferred from CoA to L-carnitine, allowing short and medium-chain acyl-carnitines to be exported from the mitochondria. This process provides free CoA needed for energy metabolism, as well as a mechanism to export excess acetyl and acyl groups from the mitochondria.

Both CoA (a critical component of beta-oxidation) and ACP function as acyl or acetyl carriers. CoA performs this function by forming thioester linkages between its sulfhydryl group and available acyl or acetyl groups. In this manner, CoA facilitates the transfer of acetyl groups from pyruvate to oxaloacetate in order to initiate the tricarboxylic acid (TCA) cycle. Before pyruvate can be used in the TCA cycle, it must be converted to acetyl-CoA by oxidative carboxylation.

The formation of fatty acids from excess amounts of glycogen involves CoA. In the first step in the synthesis of fatty acids, malonyl-CoA is formed by the carboxylation of acetyl-CoA. Fatty acid chain elongation is also dependent on CoA. The cytoplasmic fatty acid synthesizing system uses ACP, a protein analog of CoA to bind intermediates in the synthesis of long-chain fatty acids. CoA is also needed for the transport of long chain fatty acids into the mitochondria, a critical component of beta-oxidation, the process of converting fats to energy. L-carnitine plays an important role in energy production by chaperoning activated fatty acids (acyl-CoA) into the mitochondrial matrix for metabolism and chaperoning intermediate compounds out of the mitochondrial matrix to prevent their accumulation. The transport of long-chain fatty acids by L-carnitine into the mitochondrial matrix where they can be metabolized to generate energy requires three enzymes located on the mitochondrial outer and inner membranes. On the outer mitochondrial membrane of skeletal and cardiac muscle cells, carnitine-palmitoyl transferase I (CPTI) catalyzes the formation of acylcarnitine (a fatty acid+L-carnitine) from acyl-CoA (a fatty acid+coenzyme A). A transporter protein called carnitine:acylcarnitine translocase (CT) transports acylcarnitine across the inner mitochondrial membrane. Carnitine-palmitoyl transferase 11 (CPTII) is associated with the inner mitochondrial membrane and catalyzes the formation of acyl-CoA within the mitochondrial matrix where it can be metabolized through a process called beta-oxidation, ultimately yielding propionyl-CoA and acetyl-CoA.

Pancreatic beta-cell as a model: The energy requirements of most cells supplied with glucose are fulfilled by glycolytic and oxidative metabolism, yielding ATP. When cytosolic and mitochondrial contents in ATP, ADP and AMP were measured in islets incubated for 45 min at increasing concentrations of D-glucose and then exposed for 20 s to digitonin. The latter treatment failed to affect the total islet ATP/ADP ratio and adenylate charge. D-Glucose caused a much greater increase in cytosolic than mitochondrial ATP/ADP ratio. In the cytosol, a sigmoidal pattern characterized the changes in ATP/ADP ratio at increasing concentrations of D-glucose. These findings are compatible with the view that cytosolic ATP participates in the coupling of metabolic to ionic events in the process of nutrient-induced insulin release.

To gain insight into the regulation of pancreatic beta-cell mitochondrial metabolism, the direct effects on respiration of different mitochondrial substrates, variations in the ATP/ADP ratio and free Ca2+ were examined using isolated mitochondria and permeabilized clonal pancreatic beta-cells (HIT). Respiration from pyruvate was highand not influenced by Ca2+ in State 3 or under various redox states and fixed values of the ATP/ADP ratio; nevertheless, high Ca2+ elevated pyridine nucleotide fluorescence, indicating activation of pyruvate dehydrogenase by Ca2+. Furthermore, in the presence of pyruvate, elevated Ca2+ stimulated CO₂ production from pyruvate, increased citrate production and efflux from the mitochondria and inhibited CO₂ production from palmitate. The latter observation suggests that beta-cell fatty acid oxidation is not regulated exclusively by malonyl-CoA but also by the mitochondrial redox state. alpha-Glycerophosphate (alpha-GP) oxidation was Ca(2+)-dependent with a half-maximal rate observed at around 300 nM Ca2+. It was recently demonstrated that increases in respiration precede increases in Ca2+ in glucose-stimulated clonal pancreatic beta-cells (HIT), indicating that Ca2+ is not responsible for the initial stimulation of respiration. It is suggested that respiration is stimulated by increased substrate (alpha-GP and pyruvate) supply together with oscillatory increases in ADP.

The rise in Ca2+, which in itself may not significantly increase net respiration, could have the important functions of: (1) activating the alpha-GP shuttle, to maintain an oxidized cytosol and high glycolytic flux; (2) activating pyruvate dehydrogenase, and indirectly pyruvate carboxylase, to sustain production of citrate and hence the putative signal coupling factors, malonyl-CoA and acyl-CoA; (3) increasing mitochondrial redox state to implement the switch from fatty acid to pyruvate oxidation.

Glucose-stimulated increases in mitochondrial metabolism are generally thought to be important for the activation of insulin secretion. Pyruvate dehydrogenase (PDH) is a key regulatory enzyme, believed to govern the rate of pyruvate entry into the citrate cycle. It has been shown that elevated glucose concentrations (16 or 30 vs 3 mM) cause an increase in PDH activity in both isolated rat islets, and in a clonal beta-cell line (MIN6). However, increases in PDH activity elicited with either dichloroacetate, or by adenoviral expression of the catalytic subunit of pyruvate dehydrogenase phosphatase, were without effect on glucose-induced increases in mitochondrial pyridine nucleotide levels, or cytosolic ATP concentration, in MIN6 cells, and insulin secretion from isolated rat islets. Similarly, the above parameters were unaffected by blockade of the glucose-induced increase in PDH activity by adenovirus-mediated over-expression of PDH kinase (PDK). Thus, activation of the PDH complex plays an unexpectedly minor role in stimulating glucose metabolism and in triggering insulin release.

In pancreatic beta-cells, a rise in cytosolic ATP is also a critical signaling event, coupling closure of ATP-sensitive K⁺ channels (KATP) to insulin secretion via depolarization-driven increases in intracellular Ca2+. Glycolytic but not Krebs cycle metabolism of glucose is critically involved in this signaling process. While inhibitors of glycolysis suppressed glucose-stimulated insulin secretion, blockers of pyruvate transport or Krebs cycle enzymes were without effect. While pyruvate was metabolized in islets to the same extent as glucose, it produced no stimulation of insulin secretion and did not block KATP.

In pancreatic beta-cells, methyl pyruvate is a potent secretagogue and is widely used to study stimulus-secretion coupling. MP stimulated insulin secretion in the absence of glucose, with maximal effect at 5 mM. MP depolarized the beta-cell in a concentration-dependent manner (5-20 mM). Pyruvate failed to initiate insulin release (5-20 mM) or to depolarize the membrane potential. ATP production in isolated beta-cell mitochondria was detected as accumulation of ATP in the medium during incubation in the presence of malate or glutamate in combination with pyruvate or MP. ATP production by MP and glutamate was higher than that induced by pyruvate/glutamate. Pyruvate (5 mM) or MP (5 mM) had no effect on the ATP/ADP ratio in whole islets, whereas glucose (20 mM) significantly increased the whole islet ATP/ADP ratio.

In contrast with pyruvate, which barely stimulates insulin secretion, methyl pyruvate was suggested to act as an effective mitochondrial substrate. Methyl pyruvate elicited electrical activity in the presence of 0.5 mM glucose, in contrast with pyruvate. Accordingly, methyl pyruvate increased the cytosolic free Ca(2+) concentration after an initial decrease, similar to glucose. However, in contrast with glucose, methyl pyruvate even slightly decreased NAD(P)H autofluorescence and did not influence ATP production or the ATP/ADP ratio. Therefore, MP-induced beta-cell membrane depolarization or insulin release does not relate directly to mitochondrial ATP production.

The finding that methyl pyruvate directly inhibited a cation current across the inner membrane of Jurkat T-lymphocyte mitochondria suggests that this metabolite may increase ATP production in beta-cells by activating the respiratory chains without providing reduction equivalents. This mechanism may account for a slight and transient increase in ATP production. Furthermore methyl pyruvate inhibited the K(ATP) current measured in the standard whole-cell configuration. Accordingly, single-channel currents in inside-out patches were blocked by methyl pyruvate. Therefore, the inhibition of K(ATP) channels, and not activation of metabolism, mediates the induction of electrical activity in pancreatic beta-cells by methyl pyruvate.

As a membrane-permeant analog, methyl pyruvate, produced a block of KATP, a sustained rise in [Ca2+]i, and an increase in insulin secretion 6-fold the magnitude of that induced by glucose. This indicates that ATP derived from mitochondrial pyruvate metabolism does not substantially contribute to the regulation of KATP responses to a glucose challenge. Supporting the notion of sub-compartmentation of ATP within the beta-cell. Supra-normal stimulation of the Krebs cycle by methyl pyruvate can, however, overwhelm intracellular partitioning of ATP and thereby drive insulin secretion.

The metabolism of methyl pyruvate was compared to that of pyruvate in isolated rat pancreatic islets. Methyl pyruvate was found to be more efficient than pyruvate in supporting the intramitochondrial conversion of pyruvate metabolites to amino acids, inhibiting D-[5-3H]glucose utilization, maintaining a high ratio between D-[3,4-14C] glucose or D-[6-14C]glucose oxidation and D-[5-3H]glucose utilization, inhibiting the intramitochondrial conversion of glucose-derived 2-keto acids to their corresponding amino acids, and augmenting 14CO2 output from islets prelabeled with L-[U-14C] glutamine. Methyl pyruvate also apparently caused a more marked mitochondrial alkalinization than pyruvate, as judged from comparisons of pH measurements based on the use of either a fluorescein probe or 14C-labeled 5,5-dimethyl-oxazolidine-2,4-dione. Inversely, pyruvate was more efficient than methyl pyruvate in increasing lactate output and generating L-alanine. These converging findings indicate that, by comparison with exogenous pyruvate, its methyl ester is preferentially metabolized in the mitochondrial, rather than cytosolic, domain of islet cells. It is proposed that both the positive and the negative components of methyl pyruvate insulinotropic action are linked to changes in the net generation of reducing equivalents, ATP and H+.

Methyl pyruvate was found to exert a dual effect on insulin release from isolated rat pancreatic islets. A positive insulinotropic action prevailed at low concentrations of D-glucose, in the 2.8 to 8.3 mM range, and at concentrations of the ester not exceeding 10.0 mM. It displayed features typical of a process of nutrient-stimulated insulin release, such as decreased K⁺ conductance, enhanced Ca2+ influx, and stimulation of proinsulin biosynthesis. A negative insulinotropic action of methyl pyruvate was also observed, however, at a high concentration of D-glucose (16.7 mM) and/or at a high concentration of the methyl ester (20.0 mM). It was apparently not attributable to any adverse effect of methyl pyruvate on ATP generation, but might be due to hyperpolarization of the plasma membrane. The ionic determinant(s) of the latter change was not identified. The dual effect of methyl pyruvate probably accounts for an unusual time course of the secretory response, including a dramatic and paradoxical stimulation of insulin release upon removal of the ester.

Pancreatic beta-cell metabolism was followed during glucose and pyruvate stimulation of pancreatic islets using quantitative two-photon NAD(P)H imaging. The observed redox changes, spatially separated between the cytoplasm and mitochondria, were compared with whole islet insulin secretion. As expected, both NAD(P)H and insulin secretion showed sustained increases in response to glucose stimulation. In contrast, pyruvate caused a much lower NAD(P)H response and did not generate insulin secretion. Low pyruvate concentrations decreased cytoplasmic NAD(P)H without affecting mitochondrial NAD(P)H, whereas higher concentrations increased cytoplasmic and mitochondrial levels. However, the pyruvate-stimulated mitochondrial increase was transient and equilibrated to near-base-line levels. Inhibitors of the mitochondrial pyruvate-transporter and malate-aspartate shuttle were utilized to resolve the glucose- and pyruvate-stimulated NAD(P)H response mechanisms.

These data showed that glucose-stimulated mitochondrial NAD(P)H and insulin secretion are independent of pyruvate transport but dependent on NAD(P)H shuttling. In contrast, the pyruvate-stimulated cytoplasmic NAD(P)H response was enhanced by both inhibitors. Surprisingly the malate-aspartate shuttle inhibitor enabled pyruvate-stimulated insulin secretion. These data support a model in which glycolysis plays a dominant role in glucose-stimulated insulin secretion. Based on these data, it was proposed as a mechanism for glucose-stimulated insulin secretion that includes allosteric inhibition of tricarboxylic acid cycle enzymes and pH dependence of mitochondrial pyruvate transport.

Pyridine dinucleotides (NAD and NADP) are ubiquitous cofactors involved in hundreds of redox reactions essential for the energy transduction and metabolism in all living cells. In addition, NAD also serves as a substrate for ADP-ribosylation of a number of nuclear proteins, for silent information regulator 2 (Sir2)-like histone deacetylase that is involved in gene silencing regulation, and for cyclic ADP ribose (cADPR)-dependent Ca(2+) signaling. Pyridine nucleotide adenylyltransferase (PNAT) is an indispensable central enzyme in the NAD biosynthesis pathways catalyzing the condensation of pyridine mononucleotide (NMN or NaMN) with the AMP moiety of ATP to form NAD (or NaAD).

1. In isolated pancreatic islets, pyruvate causes a shift to the left of the sigmoidal curve relating the rate of insulin release to the ambient glucose concentration. The magnitude of this effect is related to the concentration of pyruvate (5—90 mM) and, at a 30 mM concentration, is equivalent to that evoked by 2 mM-glucose.

2. In the presence of glucose 8 mM), the secretory response to pyruvate is an immediate process, displaying a biphasic pattern.

3. The insulinotropic action of pyruvate coincides with an inhibition of 45Ca efflux and a stimulation of 45Ca net uptake. The relationship between 45Ca uptake and insulin release displays its usual pattern in the presence of pyruvate.

4. Exogenous pyruvate rapidly accumulates in the islets in amounts close to those derived from the metabolism of glucose. The oxidation of [2-14C]pyruvate represents 64% of the rate of [1-14C]pyruvate decarboxylation and, at a 30 mM concentration, is comparable with that of 8 mM-[U-14C]glucose.

5. When corrected for the conversion of pyruvate into lactate, the oxidation of 30 mM-pyruvate corresponds to a net generation of about 314 pmol of reducing equivalents/120 min per islet.

6. Pyruvate does not affect the rate of glycolysis, but inhibits the oxidation of glucose. Glucose does not affect pyruvate oxidation.

7. Pyruvate (30 mM) does not affect the concentration of ATP, ADP and AMP in the islet cells.

8. Pyruvate (30 mM) increases the concentration of reduced nicotinamide nucleotides in the presence but not in the absence of glucose. A close correlation is seen between the concentration of reduced nicotinamide nucleotides and the net uptake of 45Ca.

9. Pyruvate, like glucose, modestly stimulates lipogenesis.

10. Pyruvate, in contrast with glucose, markedly inhibits the oxidation of endogenous nutrients. The latter effect accounts for the apparent discrepancy between the rate of pyruvate oxidation and the magnitude of its insulinotropic action.

11. It is concluded that the effect of pyruvate to stimulate insulin release depends on its ability to increase the concentration of reduced nicotinamide nucleotides in the islet cells.

Glucose-stimulated insulin secretion is a multi-step process dependent on cell metabolic flux. Previous studies on intact pancreatic islets used two-photon NAD(P)H imaging as a quantitative measure of the combined redox signal from NADH and NADPH (referred to as NAD(P)H). These studies showed that pyruvate, a non-secretagogue, enters-cells and causes a transient rise in NAD(P)H. To further characterize the metabolic fate of pyruvate, a one-photon flavoprotein microscopy has been developed as a simultaneous assay of lipoamide dehydrogenase (LipDH) autofluorescence. This flavoprotein is in direct equilibrium with mitochondrial NADH.

Using this method, the glucose-dose response is consistent with an increase in both NADH and NADPH. In contrast, the transient rise in NAD(P)H observed with pyruvate stimulation is not accompanied by a significant change in LipDH, which indicates that pyruvate raises cellular NADPH without raising NADH. In comparison, methyl pyruvate stimulated a robust NADH and NADPH response. These data provide new evidence that exogenous pyruvate does not induce a significant rise in mitochondrial NADH. This inability likely results in its failure to produce the ATP necessary for stimulated secretion of insulin. Overall, these data are consistent with either restricted PDH dependent metabolism or a buffering of the NADH response by other metabolic mechanisms.

Glucose metabolism in glycolysis and in mitochondria is pivotal to glucose-induced insulin secretion from pancreatic beta cells. One or more factors derived from glycolysis other than pyruvate appear to be required for the generation of mitochondrial signals that lead to insulin secretion. The electrons of the glycolysis-derived reduced form of nicotinamide adenine dinucleotide (NADH) are transferred to mitochondria through the NADH shuttle system. By abolishing the NADH shuttle function, glucose-induced increases in NADH autofluorescence, mitochondrial membrane potential, and adenosine triphosphate content were reduced and glucose-induced insulin secretion was abrogated. The NADH shuttle evidently couples glycolysis with activation of mitochondrial energy metabolism to trigger insulin secretion.

To determine the role of the NADH shuttle system composed of the glycerol phosphate shuttle and malate-aspartate shuttle in glucose-induced insulin secretion from pancreatic beta cells, mice which lack mitochondrial glycerol-3 phosphate dehydrogenase mGPDH), a rate-limiting enzyme of the glycerol phosphate shuttle were used. When both shuttles were halted in mGPDH-deficient islets treated with aminooxyacetate, an inhibitor of the malate-aspartate shuttle, glucose-induced insulin secretion was almost completely abrogated. Under these conditions, although the flux of glycolysis and supply of glucose-derived pyruvate into mitochondria were unaffected, glucose-induced increases in NAD(P)H autofluorescence, mitochondrial membrane potential, Ca2+ entry into mitochondria, and ATP content were severely attenuated.

This study provides the first direct evidence that the NADH shuttle system is essential for coupling glycolysis with the activation of mitochondrial energy metabolism to trigger glucose-induced insulin secretion and thus revises the classical model for the metabolic signals of glucose-induced insulin secretion.

Incubation of porcine carotid arteries with 0.4 mmol amino-oxyacetic acid an inhibitor of glutamate-ox-aloacetate transaminase and, hence the malate-aspartate shuttle, inhibited O2 consumption by 21%, decreased the content of phosphocreatine and inhibited activity of the tricarboxylic acid cycle. The rate of glycolysis and lactate production was increased but glucose oxidation was inhibited. These effects of amino-oxyacetic acid were accompanied by evidence of inhibition of the malate-aspartate shuttle and elevation in the cytoplasmic redox potential and NADH/NAD ratio as indicated by elevation of the concentration ratios of the lactate/pyruvate and glycerol-3-phosphate/dihydroxyacetone phosphate metabolite redox couples. Addition of the fatty acid octanoate normalized the adverse energetic effects of malate-aspartate shuttle inhibition. It is concluded that the malate-aspartate shuttle is a primary mode of clearance of NADH reducing equivalents from the cytoplasm in vascular smooth muscle. Glucose oxidation and lactate production are influenced by the activity of the shuttle. The results support the hypothesis that an increased cytoplasmic NADH redox potential impairs mitochondrial energy metabolism.

Beta-Methyleneaspartate, a specific inhibitor of aspartate aminotransferase (EC 2.6.1.1.), was used to investigate the role of the malate-aspartate shuttle in rat brain synaptosomes. Incubation of rat brain cytosol, “free” mitochondria, synaptosol, and synaptic mitochondria, with 2 mM beta-methyleneaspartate resulted in inhibition of aspartate aminotransferase by 69%, 67%, 49%, and 76%, respectively. The reconstituted malate-aspartate shuttle of “free” brain mitochondria was inhibited by a similar degree (53%).

As a consequence of the inhibition of the aspartate aminotransferase, and hence the malate-aspartate shuttle, the following changes were observed in synaptosomes: decreased glucose oxidation via the pyruvate dehydrogenase reaction and the tricarboxylic acid cycle; decreased acetylcholine synthesis; and an increase in the cytosolic redox state, as measured by the lactate/pyruvate ratio. The main reason for these changes can be attributed to decreased carbon flow through the tricarboxylic acid cycle (i.e., decreased formation of oxaloacetate), rather than as a direct consequence of changes in the NAD+/NADH ratio. Malate/glutamate oxidation in “free” mitochondria was also decreased in the presence of 2 mM beta-methyleneaspartate. This is probably a result of decreased glutamate transport into mitochondria as a result of low levels of aspartate, which are needed for the exchange with glutamate by the energy-dependent glutamate-aspartate translocator.

Aminooxyacetate, an inhibitor of pyridoxal-dependent enzymes, is routinely used to inhibit gamma-aminobutyrate metabolism. The bioenergetic effects of the inhibitor on guinea-pig cerebral cortical synaptosomes are investigated. It prevents the reoxidation of cytosolic NADH by the mitochondria by inhibiting the malate-aspartate shuttle, causing a 26 mV negative shift in the cytosolic NAD+/NADH redox potential, an increase in the lactate/pyruvate ratio and an inhibition of the ability of the mitochondria to utilize glycolytic pyruvate. The 3-hydroxybutyrate/acetoacetate ratio decreased significantly, indicating oxidation of the mitochondrial NAD+/NADH couple. The results are consistent with a predominant role of the malate-aspartate shuttle in the reoxidation of cytosolic NADH in isolated nerve terminals. Aminooxyacetate limits respiratory capacity and lowers mitochondrial membrane potential and synaptosomal ATP/ADP ratios to an extent similar to glucose deprivation.

Variations in the cytoplasmic redox potential (Eh) and NADH/NAD ratio as determined by the ratio of reduced to oxidized intracellular metabolite redox couples may affect mitochondrial energetics and alter the excitability and contractile reactivity of vascular smooth muscle. To test these hypotheses, the cytoplasmic redox state was experimentally manipulated by incubating porcine carotid artery strips in various substrates. The redox potentials of the metabolite couples [lactate]/[pyruvate]i and [glycerol 3-phosphate]/[dihydroxyacetone phosphate]i varied linearly (r=0.945), indicating equilibrium between the two cytoplasmic redox systems and with cytoplasmic NADH/NAD. Incubation in physiological salt solution (PSS) containing 10 mm pyruvate ([lact]/[pyr]=0.6) increased O₂ consumption approximately 45% and produced anaplerosis of the tricarboxylic acid (TCA cycle), whereas incubation with 10 mm lactate-PSS ([lact]/[pyr]i=47) was without effect. A hyperpolarizing dose of external KCl (10 mM) produced a decrease in resting tone of muscles incubated in either glucose-PSS (−0.8+/−0.8 g) or pyruvate-PSS (−2.1+/−0.8 g), but increased contraction in lactate-PSS (1.5+/−0.7 g) (n=12-18, P<0.05). The rate and magnitude of contraction with 80 mm KCl (depolarizing) was decreased in lactate-PSS (P=0.001). Slopes of KCl concentration-response curves indicated pyruvate>glucose>lactate (P<0.0001); EC50 in lactate (29.1+/−1.0 mM) was less than that in either glucose (32.1+/−0.9 mm) or pyruvate (32.2+/−1.0 mM), P<0.03. The results are consistent with an effect of the cytoplasmic redox potential to influence the excitability of the smooth muscle and to affect mitochondrial energetics.

The cytoplasmic NADH/NAD redox potential affects energy metabolism and contractile reactivity of vascular smooth muscle. NADH/NAD redox state in the cytosol is predominately determined by glycolysis, which in smooth muscle is separated into two functionally independent cytoplasmic compartments, one of which fuels the activity of Na(+)-K(+)-ATPase. The effect was examined of varying the glycolytic compartments on cystosolic NADH/NAD redox state. Inhibition of Na(+)-K(+)-ATPase by 10 microM ouabain resulted in decreased glycolysis and lactate production. Despite this, intracellular concentrations of the glycolytic metabolite redox couples of lactate/pyruvate and glycerol-3-phosphate/dihydroxyacetone phosphate (thus NADH/NAD) and the cytoplasmic redox state were unchanged. The constant concentration of the metabolite redox couples and redox potential was attributed to: 1) decreased efflux of lactate and pyruvate due to decreased activity of monocarboxylate B—H(+) transporter secondary to decreased availability of H(+) for cotransport and; 2) increased uptake of lactate (and perhaps pyruvate) from the extracellular space, probably mediated by the monocarboxylate-H(+) transporter, which was specifically linked to reduced activity of Na(+)-K(+)-ATPase.

It was concluded that redox potentials of the two glycolytic compartments of the cytosol maintain equilibrium and that the cytoplasmic NADH/NAD redox potential remains constant in the steady state despite varying glycolytic flux in the cytosolic compartment for Na(+)-K(+)-ATPase.

Methyl pyruvate as a PPAR agonist: Peroxisomal proliferator-activated receptors (PPARs) belong to a nuclear receptor superfamily of ligand-activated transcription factors. Peroxisome proliferator-activated receptor (PPAR) is activated when a ligand binds to the ligand-binding domain at the side of C-termini. So far, three types of isoforms of alpha form, gamma form and delta form have been identified as PPARs, and the expression tissues and the functions are different respectively. Peroxisome proliferators are a structurally diverse group of compounds which, when administered to rodents, elicit dramatic increases in the size and number of hepatic and renal peroxisomes, as well as concomitant increases in the capacity of peroxisomes to metabolize fatty acids via increased expression of the enzymes required for the beta-oxidation cycle It is known that the alpha-isoform of peroxisome proliferator-activated receptor (PPAR.alpha) acts to stimulate peroxisomal proliferation in the rodent liver which leads to enhanced fatty oxidation by this organ. (PPAR) alpha is a nuclear receptor that is mainly expressed in tissues with a high degree of fatty acid oxidation such as liver, heart, and skeletal muscle. There is a sex difference in PPARalpha expression. Male rats have higher levels of hepatic PPARalpha mRNA and protein than female rats. Chemicals included in this group are the fibrate class of hypolipidermic drugs, herbicides, and phthalate plasticizers. Peroxisome proliferation can also be elicited by dietary or physiological factors such as a high-fat diet and cold acclimatization. The importance of peroxisomes in humans is stressed by the existence of a group of genetic diseases in man in which one or more peroxisomal functions are impaired. Most of the functions known to take place in peroxisomes have to do with lipids. Indeed, peroxisomes are capable of 1. fatty acid beta-oxidation 2. fatty acid alpha-oxidation 3. synthesis of cholesterol and other isoprenoids 4. ether-phospholipid synthesis and 5. biosynthesis of polyunsaturated fatty acids.

In animal cells peroxisomes as well as mitochondria are capable of degrading lipids via beta-oxidation. Nevertheless, there are important differences between the two systems. 1) The peroxisomal and mitochondrial beta-oxidation enzymes are different proteins. 2) Peroxisomal beta-oxidation does not degrade fatty acids completely but acts as a chain-shortening system, catalyzing only a limited number of beta-oxidation cycles. 3) Peroxisomal beta-oxidation is not coupled to oxidative phosphorylation and is thus less efficient than mitochondrial beta-oxidation as far as energy conservation is concerned. 4) Peroxisomal beta-oxidation is not regulated by malonyl-CoA and—as a consequence—by feeding as opposed to starvation.

Peroxisomes are responsible for the beta-oxidation of very long chain (>C20) fatty acids, dicarboxylic fatty acids, 2-methyl-branched fatty acids, prostaglandins, leukotrienes, and the carboxyl side chains of certain xenobiotics and of the bile acid intermediates di- and tri-hydroxycoprostanic acids. Mitochondria oxidize mainly long (C16-C20) chain fatty acids, which—because of their abundance—constitute a major source of metabolic fuel. The first step in peroxisomal beta-oxidation is catalyzed by two acyl-CoA oxidases in extrahepatic tissues and by three acyl-CoA oxidases in liver, each enzyme having its own substrate specificity. Palmitoyl-CoA oxidase and pristanoyl-CoA oxidase are found in liver and extrahepatic tissues. The former enzyme oxidizes the CoA esters of straight chain fatty acids, dicarboxylic fatty acids and prostaglandins; the latter enzyme oxidizes the CoA esters of branched fatty acids but also shows some activity towards straight chain and dicarboxylic fatty acids. Hepatic peroxisomes contain a third acyl-CoA oxidase, trihydroxycoprostanoyl-CoAA oxidase, which oxidizes the CoA esters of the bile acid intermediates di- an trihydroxycoprostanic acids. Treatment of rodents with a number of structurally diverse compounds called peroxisome proliferators, results in the proliferation of peroxisomes, especially in liver, and in the induction of the hepatic peroxisomal beta-oxidation enzymes except pristanoyl-CoA oxidase and trihydroxycoprostanoyl —CoA oxidase. There exist several inborn errors, in which peroxisomal beta-oxidation is deficient. These diseases are characterized by severe neurological symptoms. The biochemical findings in these diseases confirm the function of peroxisomal beta-oxidation as described above.

Insight into the mechanism whereby peroxisome proliferators exert their pleiotropic effects was provided by the identification of a member of the nuclear hormone receptor superfamily activated by these chemicals. This receptor, termed peroxisome proliferator activated receptor alpha (PPAR alpha), was subsequently shown to be activated by a variety of medium and long-chain fatty acids and to stimulate expression of the genes. The PPAR alpha binds to promoter domain of key enzymes concerning in the lipid catabolism system such as acyl-CoA synthase existing in the cytosol, acyl-CoA dehydrogenase and HMG-CoA synthase existing in the mitochondria and acyl-CoA oxidase existing in the peroxisome of liver. From the analysis of PPAR alpha-deficient mice, it is being considered that the PPAR alpha plays an important role for the energy acquisition in starvation state, that is, oxidation of fatty acid and formation of ketone body in liver.

Since the discovery of PPAR alpha additional isoforms of PPAR have been identified, PPAR beta, PPAR gamma and PPAR delta, which are spatially differentially expressed.

The nuclear peroxisome proliferator-activated receptor gamma (PPARgamma) activates the transcription of multiple genes involved in intra- and extracellular lipid metabolism. These PPARs regulate expression of target genes by binding to DNA sequence elements, termed PPAR response elements (PPRE). To date, PPRE's have been identified in the enhancers of a number of genes encoding proteins that regulate lipid metabolism suggesting that PPARs play a pivotal role in the adipogenic signaling cascade and lipid homeostasis. Because there are several isoforms of PPAR, it is desirable to identify compounds which are capable of selectively interacting with only one of the PPAR isoforms. Hypolipidaemic agents have the ability to stimulate PPAR alpha and the ensuing stimulation of peroxisomal proliferation and consequent fatty acid oxidation can account for the reduction in plasma fatty acids. PPAR-gamma plays a key role in adipocyte differentiation and insulin sensitivity—its selective synthetic ligands, the thiazolidinediones (TZD), are used as insulin sensitizers in the treatment of type 2 diabetes. Compounds also exist which exhibit agonist activity at both PPAR alpha and PPAR gamma and would be particularly effective for the treatment of obesity as well as for the treatment of diabetes/pre-diabetic insulin resistance syndrome and the resulting complications thereof. Function of PPAR delta is not very understood compared with alpha form or gamma form.

Knowledge of the mechanisms that regulate PDC activity is important, because PDC inactivation is crucial for glucose conservation when glucose is scarce, whereas adequate PDC activity is required to allow both ATP and FA production from glucose. Fuel metabolism is highly regulated to ensure adequate energy for cellular function. The contribution of the major metabolic fuels—glucose, lactate and fatty acids (FAs)—often reflects their circulating levels. In addition, regulatory cross-talk and fuel-induced hormone secretion ensures appropriate and co-ordinate fuel utilization. Because its activity can either determine or reflect fuel preference (carbohydrate versus fat), the pyruvate dehydrogenase complex (PDC) occupies a pivotal position in fuel cross-talk. Active PDC permits glucose oxidation and allows the formation of mitochondrially derived intermediates (e.g. malonyl-CoA and citrate) that reflect fuel abundance. FA oxidation suppresses PDC activity. PDC inactivation by phosphorylation is catalysed by pyruvate dehydrogenase kinases (PDKs) 1-4, which are regulated differentially by metabolite effectors. Most tissues contain at least two and often three of the PDK isoforms. A hypothesis was developed that PDK4 is a “lipid status”-responsive PDK isoform facilitating FA oxidation and signalling through citrate formation. Substrate interactions at the level of gene transcription extend glucose-FA interactions to the longer term. Isoform-specific differences in kinetic parameters, regulation, and phosphorylation site specificity of the PDKs introduce variations in the regulation of PDC activity in differing endocrine and metabolic states. Thus potential targets for substrate-mediated transcriptional regulation in relation to selective PDK isoform expression and the influence of altered PDK isoform expression in fuel sensing, selection and utilization.

Adequate flux through PDC is important in tissues with a high ATP requirement, in lipogenic tissues (since it provides cytosolic acetyl-CoA for fatty acid (FA) synthesis), and in generating cytosolic malonyl-CoA, a potent inhibitor of carnitine palmitoyltransferase (CPT I). Conversely, suppression of PDC activity is crucial for glucose conservation when glucose is scarce. Recent advances relating to the control of mammalian PDC activity by phosphorylation (inactivation) and dephosphorylation (activation, reactivation), in particular regulation of PDC by pyruvate dehydrogenase kinase (PDK) which phosphorylates and inactivates PDC. Inactivation of PDC by increased PDK activity promotes gluconeogenesis by conserving three-carbon substrates. PDK activity is that of a family of four proteins (PDK1-4). PDK2 and PDK4 appear to be expressed in most major tissues and organs of the body, PDK1 appears to be limited to the heart and pancreatic islets, and PDK3 is limited to the kidney, brain and testis. PDK4 is selectively upregulated in the longer term in most tissues and organs in response to starvation and hormonal imbalances such as insulin resistance, diabetes mellitus and hyperthyroidism. Parallel increases in PDK2 and PDK4 expression appear to be restricted to gluconceogenesic tissues, liver and kidney, which take up as well as generate pyruvate.

Immunoblot analysis with antibodies raised against recombinant PDK isoforms demonstrated changes in PDK isoform expression in response to experimental hyperthyroidism (100 microg/100 g body weight; 3 days) that was selective for fast-twitch vs slow-twitch skeletal muscle in that PDK2 expression was increased in the fast-twitch skeletal muscle (the anterior tibialis) (by 1.6-fold; P<0.05) but not in the slow-twitch muscle (the soleus). PDK4 protein expression was increased by experimental hyperthyroidism in both muscle types, there being a greater response in the anterior tibialis (4.2-fold increase; P<0.05) than in the soleus (3.2-fold increase; P<0.05). The hyperthyroidism-associated up-regulation of PDK4 expression was observed in conjunction with suppression of skeletal-muscle PDC activity, but not suppression of glucose uptake/phosphorylation, as measured in vivo in conscious unrestrained rats (using the 2-[(3)H]deoxyglucose technique). It has been proposed that increased PDK isoform expression contributes to the pathology of hyperthyroidism and to PDC inactivation by facilitating the operation of the glucose—>lactate—>glucose (Cori) and glucose—>alanine—>glucose cycles. It was also proposed that enhanced relative expression of the pyruvate-insensitive PDK isoform (PDK4) in skeletal muscle in hyperthyroidism uncouples glycolytic flux from pyruvate oxidation, sparing pyruvate for non-oxidative entry into the tricarboxylic acid (TCA) cycle, and thereby supporting entry of acetyl-CoA (derived from fatty acid oxidation) into the TCA cycle.

Regulation of PDC determines and reflects substrate preference and is critical to the ‘glucose-fatty acid cycle’, a concept of reciprocal regulation of lipid and glucose oxidation to maintain glucose homoeostasis. Mammalian PDC activity is inactivated by phosphorylation by the PDKs (pyruvate dehydrogenase kinases). PDK inhibition by pyruvate facilitates PDC activation, favouring glucose oxidation and malonyl-CoA formation: the latter suppresses LCFA (long-chain fatty acid) oxidation. PDK activation by the high mitochondrial acetyl-CoA/CoA and NADH/NAD(+) concentration ratios that reflect high rates of LCFA oxidation causes blockade of glucose oxidation. Complementing glucose homoeostasis in health, fuel allostasis, i.e. adaptation to maintain homoeostasis, is an essential component of the response to chronic changes in glycaemia and lipidaemia in insulin resistance. The concept that the PDKs act as tissue homoeostats, suggests that long-term modulation of expression of individual PDKs, particularly PDK4, is an essential component of allostasis to maintain homoeostasis. This also describes the intracellular signals that govern the expression of the various PDK isoforms, including the roles of the peroxisome proliferator-acivated receptors and lipids, as effectors within the context of allostasis.

Agonists of peroxisome proliferator-activated receptors (PPARs) have emerged as important pharmacological agents for improving insulin action. A major mechanism of action of PPAR agonists is thought to involve the alteration of the tissue distribution of nonesterified fatty acid (NEFA) uptake and utilization. To test this hypothesis directly, the effect of the novel PPARa/g agonist tesaglitazar was examined on whole-body insulin sensitivity and NEFA clearance into epididymal white adipose tissue (WAT), red gastrocnemius muscle, and liver in rats with dietary-induced insulin resistance. Wistar rats were fed a high-fat diet (59 of calories as fat) for 3 wk with or without treatment with tesaglitazar (1 mmol.kg-1.d-1, 7 d). NEFA clearance was measured using the partially metabolizable NEFA tracer, 3H—R-bromopalmitate, administered under conditions of basal or elevated NEFA availability. Tesaglitazar improved the insulin sensitivity of high-fat-fed rats, indicated by an increase in the glucose infusion rate during hyperinsulinemic-euglycemic clamp (P<0.01). This improvement in insulin action was associated with decreased diglyceride (P<0.05) and long chain acyl coenzyme A (P<0.05) in skeletal muscle. NEFA clearance into WAT of high-fat-fed rats was increased 52 by tesaglitazar under basal conditions (P<0.001). In addition the PPARa/g agonist moderately increased hepatic and muscle NEFA utilization and reduced hepatic triglyceride accumulation (P<0.05). This study shows that tesaglitazar is an effective insulin-sensitizing agent in a mild dietary model of insulin resistance. Furthermore, wthisrovide tshe first direct in vivo evidence that an agonist of both PPARa and PPARg increases the ability of WAT, liver, and skeletal muscle to use fatty acids in association with its beneficial effects on insulin action in this model.

Liver contains two pyruvate dehydrogenase kinases (PDKs), namely PDK2 and PDK4, which regulate glucose oxidation through inhibitory phosphorylation of the pyruvate dehydrogenase complex (PDC). Starvation increases hepatic PDK2 and PDK4 protein expression, the latter occurring, in part, via a mechanism involving peroxisome proliferator-activated receptor-alpha (PPARalpha). High-fat feeding and hyperthyroidism, which increase circulating lipid supply, enhance hepatic PDK2 protein expression, but these increases are insufficient to account for observed increases in hepatic PDK activity. Enhanced expression of PDK4, but not PDK2, occurs in part via a mechanism involving PPAR-alpha.

Fatty acid metabolism is transcriptionally regulated by two reciprocal systems: peroxisome proliferator-activated receptor (PPAR) a controls fatty acid degradation, whereas sterol regulatory element-binding protein-1c activated by liver X receptor (LXR) regulates fatty acid synthesis. To explore potential interactions between LXR and PPAR, the effect of LXR activation on PPARa signaling was investigated. In luciferase reporter gene assays, overexpression of LXRa or b suppressed PPARa-induced peroxisome proliferator response element-luciferase activity in a dose-dependent manner. LXR agonists, T0901317 and 22 (R)-hydroxycholesterol, dose dependently enhanced the suppressive effects of LXRs. Gel shift assays demonstrated that LXR reduced binding of PPARa/retinoid X receptor (RXR) a to peroxisome proliferator response element. Addition of increasing amounts of RXRa restored these inhibitory effects in both luciferase and gel shift assays, suggesting the presence of RXRa competition. In vitro protein binding assays demonstrated that activation of LXR by an LXR agonist promoted formation of LXR/RXRa and, more importantly, LXR/PPARa heterodimers, leading to a reduction of PPARa/RXRa formation. Supportively, in vivo administration of the LXR ligand to mice and rat primary hepatocytes substantially decreased hepatic mRNA levels of PPARa-targeted genes in both basal and PPARa agonist-induced conditions. The amount of nuclear PPARa/RXR heterodimers in the mouse livers was induced by treatment with PPARa ligand, and was suppressed by superimposed LXR ligand. Taken together with data from the paper (Yoshikawa, T., T. Ide, H. Shimano, N. Yahagi, M. Amemiya-Kudo, T. Matsuzaka, S. Yatoh, T. Kitamine, H. Okazaki, Y. Tamura, M. Sekiya, A. Takahashi, A. H. Hasty, R. Sato, H. Sone, J. Osuga, S. Ishibashi, and N. Yamada, Endocrinology 144:1240-1254) describing PPARa suppression of the LXR-sterol regulatory element-binding protein-1c pathway, it has been proposed that the presence of an intricate network of nutritional transcription factors with mutual interactions, resulting in efficient reciprocal regulation of lipid degradation and lipogenesis.

Heterodimerization partners for retinoid X receptors (RXRs) include PPARalpha and thyroid-hormone receptors (TRs). The responses were investigated of hepatic PDK protein expression to high-fat feeding and hyperthyroidism in relation to hepatic lipid delivery and disposal. High-fat feeding increased hepatic PDK2, but not PDK4, protein expression whereas hyperthyroidism increased both hepatic PDK2 and PDK4 protein expression. Both manipulations decreased the sensitivity of hepatic carnitine palmitoyltransferase I (CPT I) to suppression by malonyl-CoA, but only hyperthyrodism elevated plasma fatty acid and ketone-body concentrations and CPT I maximal activity. Administration of the selective PPAR-alpha activator WY14,643 significantly increased PDK4 protein to a similar extent in both control and high-fat-fed rats, but WY14,643 treatment and hyperthyroidism did not have additive effects on hepatic PDK4 protein expression. PPAR-alpha activation did not influence hepatic PDK2 protein expression in euthyroid rats, suggesting that up-regulation of PDK2 by hyperthyroidism does not involve PPARalpha, but attenuated the effect of hyperthyroidism to increase hepatic PDK2 expression. The results indicate that hepatic PDK4 up-regulation can be achieved by heterodimerization of either PPAR alpha or TR with the RXR receptor and that effects of PPAR alpha activation on hepatic PDK2 and PDK4 expression favour a switch towards preferential expression of PDK4.

The pyruvate dehydrogenase complex (PDC) occupies a strategic role in renal intermediary metabolism, via partitioning of pyruvate flux between oxidation and entry into the gluconeogenic pathway. Inactivation of PDC via activation of pyruvate dehydrogenase kinases (PDKs), which catalyze PDC phosphorylation, occurs secondary to increased fatty acid oxidation (FAO). In kidney, inactivation of PDC after prolonged starvation is mediated by up-regulation of the protein expression of two PDK isoforms, PDK2 and PDK4. The lipid-activated transcription factor, peroxisome proliferator-activated receptor-alpha (PPAR alpha), plays a pivotal role in the cellular metabolic response to fatty acids and is abundant in kidney. In the present study PPAR alpha null mice were used to examine the potential role of PPAR alpha in regulating renal PDK protein expression. In wild-type mice, fasting (24 h) induced marked up-regulation of the protein expression of PDK4, together with modest up-regulation of PDK2 protein expression. In striking contrast, renal protein expression of PDK4 was only marginally induced by fasting in PPAR alpha null mice. The present results define a critical role for PPAR alpha in renal adaptation to fasting, and identify PDK4 as a downstream target of PPAR alpha activation in the kidney. It has been proposed that specific up-regulation of renal PDK4 protein expression in starvation, by maintaining PDC activity relatively low, facilitates pyruvate carboxylation to oxaloacetate and therefore entry of acetyl-CoA derived from FA beta-oxidation into the TCA cycle, allowing adequate ATP production for brisk rates of gluconeogenesis.

Factors that regulate PDK4 expression include FA oxidation and adequate insulin action. PDK4 is also either a direct or indirect target of peroxisome proliferator-activated receptor (PPAR) alpha. PPAR alpha deficiency in liver and kidney restricts starvation-induced upregulation of PDK4; however, the role of PPAR alpha in heart and skeletal muscle appears to be more complex. These observations may have important implications for the pharmacological modulation of PDK activity (e.g. use of PPAR alpha activators) for the control of whole-body glucose, lipid and lactate homeostasis in disease states and suggest that therapeutic interventions must be tissue targeted so that whole-body fuel homeostasis is not adversely perturbed.

Regulation of the activity of the pyruvate dehydrogenase complex in skeletal muscle plays an important role in fuel selection and glucose homeostasis. Activation of the complex promotes disposal of glucose, whereas inactivation conserves substrates for hepatic glucose production. Starvation and diabetes induce a stable increase in pyruvate dehydrogenase kinase activity in skeletal muscle mitochondria that promotes phosphorylation and inactivation of the complex. The present study shows that these metabolic conditions induce a large increase in the expression of PDK4, one of four pyruvate dehydrogenase kinase isoenzymes expressed in mammalian tissues, in the mitochondria of gastrocnemius muscle. Refeeding starved rats and insulin treatment of diabetic rats decreased pyruvate dehydrogenase kinase activity and also reversed the increase in PDK4 protein in gastrocnemius muscle mitochondria. Starvation and diabetes also increased the abundance of PDK4 mRNA in gastrocnemius muscle, and refeeding and insulin treatment again reversed the effects of starvation and diabetes. These findings suggest that an increase in amount of this enzyme contributes to hyper-phosphorylation and inactivation of the pyruvate dehydrogenase complex in these metabolic conditions. It was further found that feeding rats WY-14,643, a selective agonist for the peroxisome proliferator-activated receptor-alpha (PPAR-alpha), also induced large increases in pyruvate dehydrogenase kinase activity, PDK4 protein, and PDK4 mRNA in gastrocnemius muscle. Since long-chain fatty acids activate PPAR-alpha endogenously, increased levels of these compounds in starvation and diabetes may signal increased expression of PDK4 in skeletal muscle.

The transcriptional coactivator PPAR gamma coactivator 1 alpha (PGC-1alpha) is a key regulator of metabolic processes such as mitochondrial biogenesis and respiration in muscle and gluconeogenesis in liver. Reduced levels of PGC-1alpha in humans have been associated with type II diabetes. PGC-1alpha contains a negative regulatory domain that attenuates its transcriptional activity. This negative regulation is removed by phosphorylation of PGC-1alpha by p38 MAPK, an important kinase downstream of cytokine signaling in muscle and beta-adrenergic signaling in brown fat. Described here the identification of p160 myb binding protein (p160 MBP) as a repressor of PGC-1alpha. The binding and repression of PGC-1alpha by p160 MBP is disrupted by p38 MAPK phosphorylation of PGC-1alpha. Adenoviral expression of p160 MBP in myoblasts strongly reduces PGC-1alpha's ability to stimulate mitochondrial respiration and the expression of the genes of the electron transport system. This repression does not require removal of PGC-1alpha from chromatin, suggesting that p160 MBP is or recruits a direct transcriptional suppressor. Overall, these data indicate that p160 MBP is a powerful negative regulator of PGC-1alpha function and provide a molecular mechanism for the activation of PGC-1alpha by p38 MAPK.

It is well established that catecholamine-stimulated thermogenesis in brown fat requires beta-adrenergic elevations in cyclic AMP (cAMP) to increase expression of the uncoupling protein 1 (UCP1) gene. However, little is known about the downstream components of the signaling cascade or the relevant transcription factor targets thereof. Helt has beenemonstrate tdhat cAMP-and protein kinase A-dependent activation of p38 mitogen-activated protein kinase (MAPK) in brown adipocytes is an indispensable step in the transcription of the UCP1 gene in mice. By phosphorylating activating transcription factor 2 (ATF-2) and peroxisome proliferator-activated receptor gamma (PPARgamma) coativator 1alpha (PGC-1alpha), members of two distinct nuclear factor families, p38 MAPK controls the expression of the UCP1 gene through their respective interactions with a cAMP response element and a PPAR response element that both reside within a critical enhancer motif of the UCP1 gene. Activation of ATF-2 by p38 MAPK additionally serves as the cAMP sensor that increases expression of the PGC-1alpha gene itself in brown adipose tissue. In conclusion, outheseindings illustrate that by orchestrating the activity of multiple transcription factors, p38 MAPK is a central mediator of the cAMP signaling mechanism of brown fat that promotes thermogenesis.

Brown adipose tissue expresses the thermogenic uncoupling protein-1 (UCP-1), which is positively regulated by peroxisome proliferator-activated receptor (PPAR) agonists and retinoids through the activation of the heterodimers PPAR/retinoid X receptor (RXR) and retinoic acid receptor (RAR)/RXR and binding to specific elements in the ucp-1 enhancer. In a study it was shown that in fetal rat brown adipocyte primary cultures the PPARgamma agonist rosiglitazone (Rosi), as well as retinoic acids 9-cis-retinoic acid and all-trans-retinoic acid also have “extragenic” effects and induce p44/p42 and p38 mitogen-activated protein kinase (p38MAPK) activation. The latter is involved in UCP-1 gene expression, because inhibition of p38MAPK activity with PD169316 impairs the ability of Rosi and retinoids for UCP-1 induction. The inhibitory effects of PD169316 are mimicked by the antioxidant GSH, suggesting a role for reactive oxygenated species (ROS) generation in the increase of UCP-1 expression in response either to Rosi or 9-cis-retinoic acid. Thus, it was proposed that Rosi and retinoids act as PPAR/RXR and RAR/RXR agonists and also activate p38MAPK. These two coordinated actions could result in a high increase of transcriptional activity on the ucp-1 enhancer and hence on thermogenesis. PPARalpha and gamma agonists but not retinoids also increase UCP-3 expression in fetal brown adipocytes. However, the regulation of UCP-3, which is not involved in thermogenesis, seems to differ from UCP-1 given the fact that is not affected by p38MAPK inhibition.

Brown adipose tissue is present in rodents but not in adult humans. It expresses uncoupling protein 1 (UCP1) that allows dissipation of energy as heat. Peroxisome proliferator-activated receptor gamma (PPARgamma) and PPARgamma coactivator 1alpha (PGC-1alpha) activate mouse UCP1 gene transcription. It has been shown that human PGC-1alpha induced the activation of the human UCP1 promoter by PARgamma. Adenovirus-mediated expression of human PGC-1alpha increased the expression of UCP1, respiratory chain proteins, and fatty acid oxidation enzymes in human subcutaneous white adipocytes. Changes in the expression of other genes were also consistent with brown adipocyte mRNA expression profile. PGC-1alpha increased the palmitate oxidation rate by fat cells. Human white adipocytes can therefore acquire typical features of brown fat cells. The PPARgamma agonist rosiglitazone potentiated the effect of PGC-1alpha on UCP1 expression and fatty acid oxidation. Hence, PGC-1alpha is able to direct human WAT PPARgamma toward a transcriptional program linked to energy dissipation. However, the response of typical white adipocyte targets to rosiglitazone treatment was not altered by PGC-1alpha. UCP1 mRNA induction was shown in vivo by injection of the PGC-1alpha adenovirus in mouse white fat. Alteration of energy balance through an increased utilization of fat in WAT may be a conceivable strategy for the treatment of obesity.

Extracellular regulated kinases (ERKs) mediate the inhibitory effect of tumor necrosis factor alpha (TNF-alpha) on uncoupling protein-1 (UCP-1), but not on lipid accumulation. TNF-alpha-induced ERK-dependent peroxisome proliferator activator receptor gamma (PPAR gamma) phosphorylation could be responsible for UCP-1 downregulation. Thus, the negative effect of TNF-alpha on UCP-1 mRNA expression at 4-5 h, under basal conditions or in cells treated with the PPAR gamma agonist, rosiglitazone, was reversed by the MEK1 inhibitor PD98059. In contrast, fatty acid synthase and malic enzyme mRNA downregulation was not prevented. Moreover, rosiglitazone has no positive effect on adipogenic gene expression or lipid accumulation. Therefore, there is a differential regulation of thermogenic and adipogenic differentiation by PPAR gamma, which might account for the differences in the TNF-alpha regulation through ERKs.

In rat pancreatic islets chronically exposed to high glucose or high free fatty acid (FFA) levels, glucose-induced insulin release and mitochondrial glucose oxidation are impaired. These abnormalities are associated with high basal ATP levels but a decreased glucose-induced ATP production (Delta of increment over baseline 0.7+/−0.5 or 0.5 +/−0.3 pmol/islet in islets exposed to glucose or FFA vs. 12.0+/−0.6 in control islets, n=3; P<0.01) and, as a consequence, with an altered ATP/ADP ratio. To investigate further the mechanism of the impaired ATP form ation, in rat pancreatic islets glucose-stimulated pyruvate dehydrogenase (PDH) activity was measured, a key enzyme for pyruvate metabolism and for the subsequent glucose oxidation through the Krebs cycle, and also the uncoupling protein-2 (UCP-2) content by Western blot. In islets exposed to high glucose or FFA, glucose-stimulated PDH activity was impaired and UCP-2 was overexpressed. Because UCP-2 expression is modulated by a peroxisome proliferator-activated receptor (PPAR)-dependent pathway, PPAR-gamma contents were measured by Western blot and the effects of a PPAR-gamma antagonist. PPAR-gamma levels were overexpressed in islets cultured with high FFA levels but unaffected in islets exposed to high glucose. In islets exposed to high FFA concentration, a PPAR-gamma antagonist was able to prevent UCP-2 over-expression and to restore insulin secretion and the ATP/ADP ratio. These data indicate that in rat pancreatic islets chronically exposed to high glucose or FFA, glucose-induced impairment of insulin secretion is associated with (and might be due to) altered mitochondrial function, which results in impaired glucose oxidation, over-expression of the UCP-2 protein, and a consequent decrease of ATP production. This alteration in FFA cultured islets is mediated by the PPAR-gamma pathway.

Methyl pyruvate has been described with reference to a particular embodiment. For one skilled in the art, other modifications and enhancements can be made without departing from the spirit and scope of the aforementioned claims.

Whilst endeavoring in the foregoing Specification to draw attention to those features of the invention believed to be of particular importance it should be understood that the Applicant claims protection in respect of any patentable feature hereinbefore referred to whether or not particular emphasis has been placed thereon.

1. We claim a method of controlling the weight or to induce a weight loss or to reduce an expected weight gain from a given diet in a mammal with the use of methyl pyruvate.

2. We claim a method of controlling the weight or to induce a weight loss or to reduce an expected weight gain from a given diet in a mammal with the use of methyl pyruvic acid.

3. We claim the method of claim 1 wherein body fat deposition in said mammal is effectively reduced by administering methyl pyruvate.

4. We claim the method of claim 2 wherein body fat deposition in said mammal is effectively reduced by administering methyl pyruvic acid.

5. We claim the method of claim 2 wherein a therapeutic and effective amount of methyl pyruvic acid is infused or orally administered to the mammal.

6. We claim the method of claim 1 wherein a therapeutic and effective amount of the salt of methyl pyruvate is infused or orally administered to the mammal.

7. We claim the method of claim 6 wherein the salt of methyl pyruvate is a monovalent cation (such as sodium or potassium methyl pyruvate).

8. We claim the method of claim 6 wherein the salt of methyl pyruvate is a divalent cation (such as calcium or magnesium methyl pyruvate).

9. We claim the method of claim 6 wherein analogs of these compounds can act as substrates or substrate analogs for methyl pyruvate.

10. We claim the method of claim 6 wherein the salt of methyl pyruvate and composition of a pharmacologically acceptable excipient and/or diluent therefor.

11. We claim the method of claim 10 wherein the salt of methyl pyruvate and composition which further comprises vitamins, coenzymes, mineral substances, amino acids, herbs, creatine compounds a, metabolic compoundsnd antioxidants.

12. We claim the method of claim 10, orally administrable, in the form of a dietary supplement or energizer or pharmaceutical drug.

13. We claim the method of claim 11, orally administrable, in the form of a dietary supplement or energizer or pharmaceutical drug.

14. We claim the method of claim 12, in the form of lozenges, tablets, pills, capsules, powders, granulates, sachets, syrups or vials.

15. We claim the method of claim 13, in the form of lozenges, tablets, pills, capsules, powders, granulates, sachets, syrups or vials.

16. We claim the method of claim 14, in unit dosage form, comprising from about 100 mg to about 28 grams of at least one of the salts, preferably about between 0.5 gram and 5 grams.

17. We claim the method of claim 15, in unit dosage form, comprising from about 100 mg to about 28 grams of at least one of the salts, preferably about between 0.5 gram and 5 grams.

18. We claim the method of claim 16 for increasing the protein concentration in the body of a mammal, which comprises administering to a mammal a therapeutically effective amount of methyl pyruvate for a period of at least about 5 days to increase body protein concentration.

19. We claim the method of claim 17 for increasing the protein concentration in the body of a mammal, which comprises administering to a mammal a therapeutically effective amount of methyl pyruvate for a period of at least about 5 days to increase body protein concentration.

20. We claim the method of claim 5 wherein analogs can act as substrates or substrate analogs for methyl pyruvic acid.

21. We claim the method of claim 5 wherein methyl pyruvic acid and composition of a pharmacologically acceptable excipient and/or diluent therefor.

22. We claim the method of claim 21 wherein methyl pyruvic acid and composition which further comprises vitamins, coenzymes, mineral substances, amino acids, herbs, creatine compounds a, metabolic compounds,nd antioxidants.

23. We claim the method of claim 21, orally administrable, in the form of a dietary supplement or energizer or pharmaceutical drug.

24. We claim the method of claim 22, orally administrable, in the form of a dietary supplement or energizer or pharmaceutical drug.

25. We claim the method of claim 23, in the form of lozenges, tablets, pills, capsules, powders, granulates, sachets, syrups or vials.

26. We claim the method of claim 24, in the form of lozenges, tablets, pills, capsules, powders, granulates, sachets, syrups or vials.

27. We claim the method of claim 25, in unit dosage form, comprising from about 100 mg to about 28 grams, preferably about between 0.5 gram and 5 grams.

28. We claim the method of claim 26, in unit dosage form, comprising from about 100 mg to about 28 grams, preferably about between 0.5 gram and 5 grams.

29. We claim the method of claim 27 for increasing the protein concentration in the body of a mammal, which comprises administering to a mammal a therapeutically effective amount of methyl pyruvic acid for a period of at least about 5 days to increase body protein concentration.

30. We claim the method of claim 28 for increasing the protein concentration in the body of a mammal, which comprises administering to a mammal a therapeutically effective amount of methyl pyruvic acid for a period of at least about 5 days to increase body protein concentration.
